(19) United States
(12) Patent Application Publication
(10) Pub. No.: US 2016/0214961 A1
(43) Pub. Date: Jul. 28, 2016

(54) NOVEL SALTS OF CRIZOTINIB AND THEIR PREPARATION

(71) Applicant: SHILPA MEDICARE LIMITED, Karnataka, Raichur (IN)

(72) Inventors: LAVKUMAR UPALLA, VIZIANAGARAM (IN); SRIRAM RAMPALLI, VIZIANAGARAM (IN); CHANTIBABU PATNEEDI, VIZIANAGARAM (IN); CHANDRASEKHAR DANGUDUBIYYAM, VIZIANAGARAM (IN); AKSHAYKANT CHATURVEDI, RAICHUR (IN)

(73) Assignee: SHILPA MEDICARE LIMITED, RAICHUR (IN)

(21) Appl. No.: 14/917,279

(22) PCT Filed: Sep. 3, 2014
(86) PCT No.: PCT/IB2014/064224

§ 371 Date: Mar. 8, 2016

(30) Foreign Application Priority Data
Sep. 10, 2013 (IN) 4026/CHE/2013

(51) Int. Cl. C07D 401/14; C07C 59/52; C07C 59/74

(52) U.S. Cl. CPC C07D 401/14; C07C 59/74; C07C 59/52

(57) ABSTRACT

The present invention relates to novel pharmaceutically acceptable substituted aryl acrylic acid addition salts of Crizotinib (I) or its hydrate or solvate thereof.

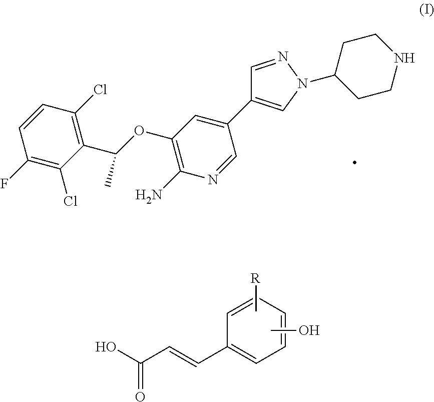

The present invention further relates to processes for preparation of the said substituted aryl acrylic acid addition salts of Crizotinib (I). The present application also provides pharmaceutically acceptable substituted aryl acrylic acid addition salts of Crizotinib (I) or its hydrate or solvate useful as active pharmaceutical ingredient in pharmaceutical composition comprising thereof, possessing anti-cancer activity.

### FIELD OF THE INVENTION

The present invention relates to novel pharmaceutically acceptable substituted aryl acrylic acid addition salts of Crizotinib (I) or its hydrate or solvate thereof.

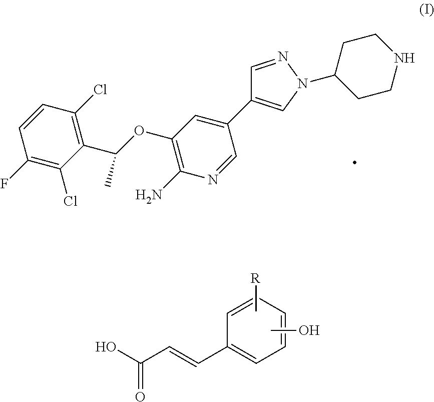

The present invention further relates to processes for preparation of the said substituted aryl acrylic acid addition salts of Crizotinib (I). The present application also provides pharmaceutically acceptable substituted aryl acrylic acid addition salts of Crizotinib (I) or its hydrate or solvate useful as active pharmaceutical ingredient in pharmaceutical composition comprising thereof, possessing anti-cancer activity.

### INTRODUCTION

Crizotinib is a kinase inhibitor indicated for the treatment of patients with locally advanced or metastatic non-small cell lung cancer (NSCLC) that is anaplastic lymphoma kinase (ALK)-positive. It was approved by USFDA in August of 2011 and is marketed under the trade name XALKORI™. Crizotinib is also undergoing clinical trials testing its safety and efficacy in anaplastic large cell lymphoma, neuroblastoma, and other advanced solid tumors in both adults and children.

Crizotinib is chemically mentioned in the USFDA label as (R)-3-[1-(2,6-Dichloro-3-fluorophenyl)ethoxy]-5-[1-(piperidin-4-yl)-1H-pyrazol-4-yl]pyridin-2-amine (I).

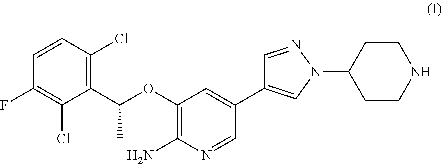 [SMILES: C[C@@H](Oc1cc(-c2cnn(C3CCNCC3)c2)cnc1N)c1c(Cl)ccc(F)c1Cl]

Crizotinib structure contains one chiral center. It is a non-hygroscopic, white to pale yellow powder which is insoluble in water, though being highly soluble in acidic pH. Crizotinib is defined in class IV (low solubility and low permeability) under the Biopharmaceutics Classification System (BCS).

Cui, et al. in U.S. Pat. No. 7,230,098 B2 provided the first generic disclosure of (R)-3-[1-(2,6-Dichloro-3-fluorophenyl)ethoxy]-5-[1-(piperidin-4-yl)-1H-pyrazol-4-yl]pyridin-2-amine (also known as Crizotinib).

Further Cui, et al. in U.S. Pat. No. 7,858,643 B2 provided the specific disclosure of Crizotinib and its pharmaceutically acceptable salts. This patent also describes the process for preparation of Crizotinib. The term “pharmaceutically acceptable salt” has been referred to include-acid addition salts, with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like. Also compounds wherein an acidic proton present in the parent compound is either replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like, have been disclosed to be amongst “pharmaceutically acceptable salts”.

Then, Cui, et al. in U.S. Pat. No. 8,217,057 B2 disclosed a crystalline form of the free base of (R)-3-[1-(2,6-dichloro-3-fluoro-phenyl)-ethoxy]-5-(1-piperidin-4-yl-1H-pyrazol-4-yl)-pyridin-2-ylamine, designated as Form 1. U.S. Pat. No. 8,217,057 B2 mentions that the crystalline form of the free base of (R)-3-[1-(2,6-dichloro-3-fluoro-phenyl)-ethoxy]-5-(1-piperidin-4-yl-H-pyrazol-4-yl)-pyridin-2-ylamine is an anhydrous or a hydrate form.

Available literature is indicative that very less is known about salt forms of Crizotinib. Choosing a pharmaceutically acceptable salt with desired combination of properties is an unpredictable activity. Selection of a suitable salt form involves an interplay of chemical properties, formulation feasibility and analysis from perspective of drug metabolism. Newer salt forms of a drug may come out as important therapeutic agents with better properties-biological as well as physical w.r.t. the base form or other salt forms. Different salt forms are generally known to offer improvement in physicochemical properties thus influencing absorption, distribution, metabolism, and excretion. It is believed that better physiochemical properties of a compound in turn affect its therapeutic efficacy as well.

Hence there appears to be need for new salts of Crizotinib which may provide advantages like further improved physical and/or chemical and/or therapeutic properties. Hence it was thought worthwhile by the inventors of the present application to explore pharmaceutically acceptable substituted aryl acrylic acid addition salts of Crizotinib, which may further improve the characteristics of drug Crizotinib.

More particularly, salts of Crizotinib with substituted aryl acrylic acid viz. Ferulic acid, Coumaric acid, Caffeic acid and the like have been explored by the inventors of the present application. Substituted aryl acrylic acids are found to occur naturally and may be obtainable by synthetic processes as well. The choice of substituted aryl acrylic acids for salt formation with Crizotinib base has been based on the premise that these salts are pharmaceutically acceptable and have favorable safety profile.

For example, Ferulic acid is a known component of ‘asafoetida’, the dried latex from the giant fennel (Ferula communis). Further, it is found in the seeds of coffee, apple, peanut, and orange, as well as in both seeds and cell walls of plants such as rice, wheat, oats, and pineapple. Ferulic acid is also found in rice bran oil, which is a popular cooking oil.

Coumaric acid, more particularly p-Coumaric acid can be found in a wide variety of edible plants such as peanuts, beans, tomatoes, carrots, and garlic. It is also found in wine and vinegar. p-Coumaric acid as glucoside can also be found in commercial breads containing flaxseed. Diesters of p-Coumaric acid can be found in carnauba wax.

Caffeic acid is found in the bark of Eucalyptus globulus. It can also be found in the fresh water fern Salvinia molesta or in the mushroom Phellinus linteus. Amongst food materials Caffeic acid is found in coffee. It is one of the main natural phenols in argan oil.

Thus, inventors of the present application provide novel pharmaceutically acceptable addition salts of substituted aryl acrylic acid with Crizotinib and process for their preparation. These new salts of Crizotinib as per present application are found to be stable and offer various advantages in terms of storage, shelf life and improved physical and/or chemical properties.

### SUMMARY OF INVENTION

Particular aspects of the present specification relate to the novel pharmaceutically acceptable substituted aryl acrylic acid addition salts of Crizotinib (I) or hydrate or solvate thereof and process for their preparation. Further, the present invention of this application also relates to pharmaceutical compositions comprising of stable pharmaceutically acceptable substituted aryl acrylic acid addition salts of Crizotinib (I) or hydrate or solvate thereof, which are useful in the treatment of various cancerous disorders.

In one aspect of the present application, the present invention provides stable pharmaceutically acceptable substituted aryl acrylic acid addition salt of Crizotinib represented by Formula (I) or a hydrate or solvate thereof,

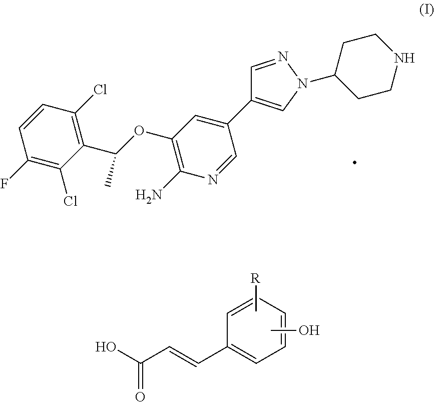

wherein, R is selected from —H, —OH or —O—C₁₋₃alkyl.

In another aspect of the present application, stable pharmaceutically acceptable substituted aryl acrylic acid addition salt of Crizotinib (I), or a hydrate or solvate thereof is optionally in crystalline form or in amorphous form.

In a further aspect of the present application, stable pharmaceutically acceptable substituted aryl acrylic acid addition salt of Crizotinib (I) is represented specifically by Formula (Ia),

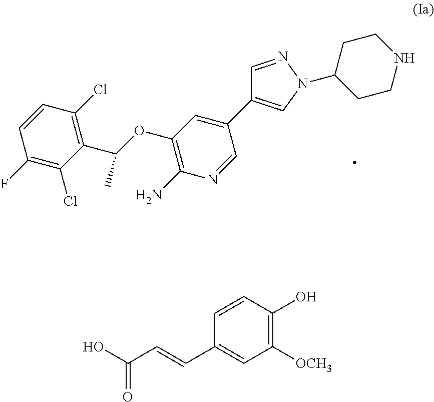 [SMILES: C[C@@H](Oc1cc(-c2cnn(C3CCNCC3)c2)cnc1N)c1c(Cl)ccc(F)c1Cl ; COc1cc(/C=C/C(=O)O)ccc1O]

and is characterized by X-ray powder diffraction pattern as per FIG. 1 and IR absorption peaks, at approximately 3377 cm⁻¹, 1321 cm⁻¹, 1593 cm⁻¹, 1634 cm⁻¹, 1124 cm⁻¹ and 776 cm⁻¹.

In another aspect of the present application, stable pharmaceutically acceptable substituted aryl acrylic acid addition salt of Crizotinib (I) is represented specifically by Formula (Ib),

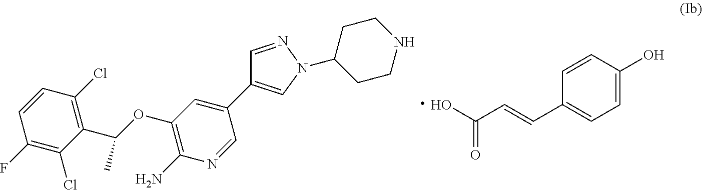 [SMILES: C[C@@H](Oc1cc(-c2cnn(C3CCNCC3)c2)cnc1N)c1c(Cl)ccc(F)c1Cl ; O=C(O)/C=C/c1ccc(O)cc1]

and is characterized by X-ray powder diffraction pattern as per FIG. 4 and IR absorption peaks, at approximately 3356 cm⁻¹, 1274 cm⁻¹, 1587 cm⁻¹, 1634 cm⁻¹, 1117 cm⁻¹ and 776 cm⁻¹.

In yet another aspect of the present application, it relates to a process for preparing stable pharmaceutically acceptable substituted aryl acrylic acid addition salt of Crizotinib (I) comprising the steps of:
a) providing a solution of Crizotinib base in an organic solvent at temperature of 20-30° C.;
b) adding substituted aryl acrylic acid of Formula (A) to the reaction mixture;

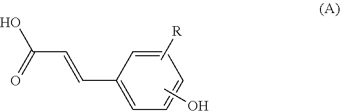

wherein, R is selected from —H, —OH, or —O—C₁₋₃alkyl;
c) extracting the solvent from reaction mixture;
d) treating the product obtained from step c) with another organic solvent;
e) isolating the stable pure crystalline pharmaceutically acceptable substituted aryl acrylic acid addition salt of Crizotinib (I).

In a further aspect of the present invention, it relates to a pharmaceutical composition comprising stable pharmaceutically acceptable substituted aryl acrylic acid addition salt of Crizotinib (I) or a hydrate or solvate thereof, together with one or more pharmaceutically acceptable excipients.

Further particular aspects of the invention are detailed in the description of invention, wherever appropriate.

### BRIEF DESCRIPTION OF THE DRAWINGS

FIG. 1 is an example of X-ray powder diffraction (“XRPD”) pattern of Crizotinib Ferulate (Ia).

FIG. 2 is an example of a ¹H NMR spectrum of Crizotinib Ferulate (Ia).

FIG. 3 is an example of IR spectral pattern of Crizotinib Ferulate (Ia).

FIG. 4 is an example of X-ray powder diffraction (“XRPD”) pattern of Crizotinib Coumarate (Ib).

FIG. 5 is an example of a ¹H NMR spectrum of Crizotinib Coumarate (Ib).

FIG. 6 is an example of IR spectral pattern of Crizotinib Coumarate (Ib).

### ABBREVIATIONS

| | |
|---|---|
| BCS | Biopharmaceutics Classification System |
| DMSO | DiMethylSulfOxide |
| d | doublet |
| dd | doublet of doublet |
| IR | Infrared Spectroscopy |
| ¹HNMR | Proton Nuclear Magnetic Resonance |
| MTBE | Methyl Tertiary-Butyl Ether |
| m | multiplet |
| q | quartet |
| s | singlet |
| XRPD | X-Ray Powder Diffraction |

### DETAILED DESCRIPTION

As set forth herein, embodiments of the present invention relate to stable pharmaceutically acceptable substituted aryl acrylic acid addition salts of Crizotinib (I) or hydrate or solvate thereof and process for their preparation.

In one embodiment of the present application, it provides stable pharmaceutically acceptable substituted aryl acrylic acid addition salt of Crizotinib represented by Formula (I) or a hydrate or solvate thereof,

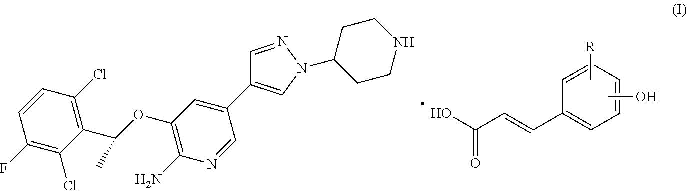

wherein, R is selected from —H, —OH or —O—C₁₋₃alkyl.

In a further embodiment of the present application, it provides that group —R in Formula (I) is selected from —H, —OH or —OCH₃. Substituted aryl acrylic acids used for salt formation with Crizotinib base can be selected from Ferulic acid, o-Coumaric acid, m-Coumaric acid, p-Coumaric acid, Caffeic acid and the like.

In another embodiment of the present application, stable pharmaceutically acceptable substituted aryl acrylic acid addition salt of Crizotinib represented by Formula (I) or a hydrate or solvate thereof may be present in crystalline or amorphous form.

In a preferred embodiment of the present invention stable pharmaceutically acceptable substituted aryl acrylic acid addition salt of Crizotinib (I) is represented specifically by Formula (Ia).

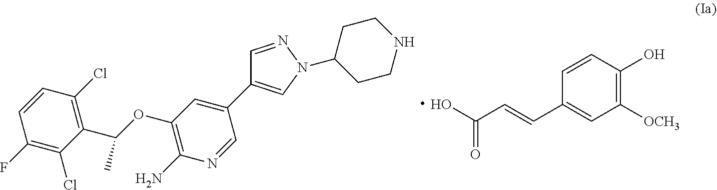 [SMILES: C[C@@H](Oc1cc(-c2cnn(C3CCNCC3)c2)cnc1N)c1c(Cl)ccc(F)c1Cl ; COc1cc(/C=C/C(=O)O)ccc1O]

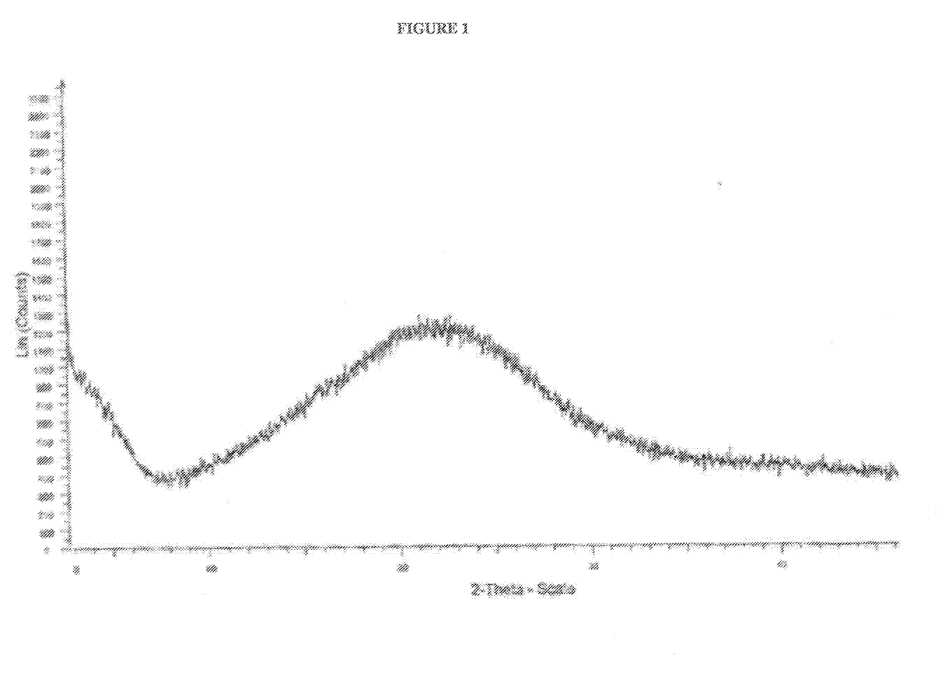

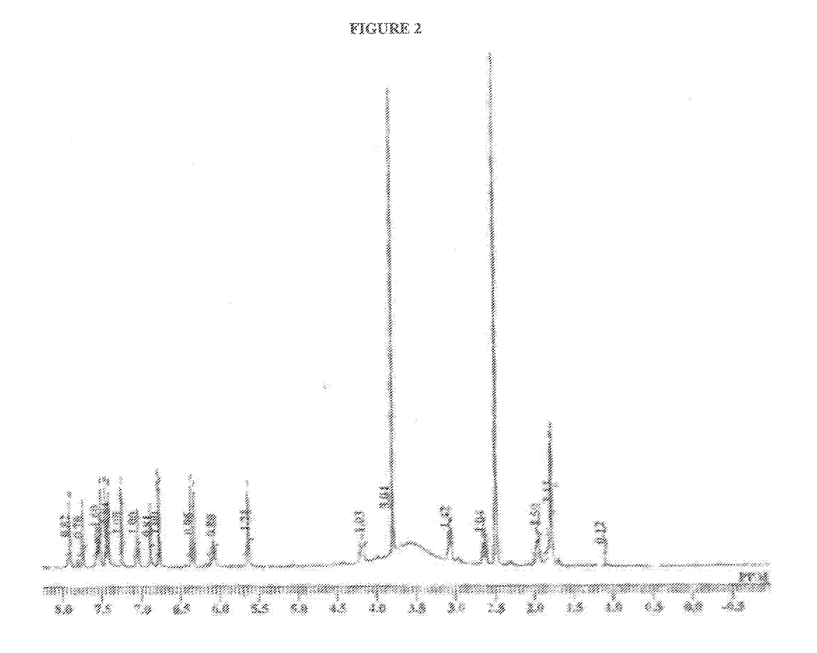

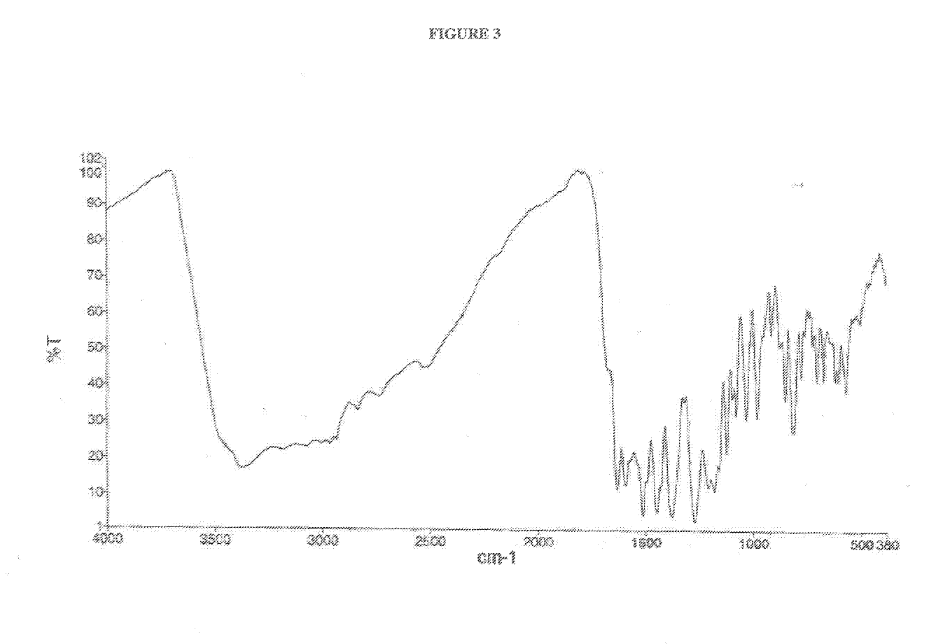

In a further embodiment of the present invention stable pharmaceutically acceptable substituted aryl acrylic acid addition salt of Crizotinib represented by Formula (Ia) i.e. Crizotinib Ferulate is characterized by:
a) X-ray powder diffraction pattern substantially according to FIG. 1;
b) ¹H NMR spectrum substantially according to FIG. 2;
c) IR absorption peaks, at approximately 3377 cm⁻¹, 1321 cm⁻¹, 1593 cm⁻¹, 1634 cm⁻¹, 1124 cm⁻¹ and 776 cm⁻¹ or IR spectral pattern substantially according to FIG. 3.

In another preferred embodiment of the present invention stable pharmaceutically acceptable substituted aryl acrylic acid addition salt of Crizotinib (I) is represented specifically by Formula (Ib).

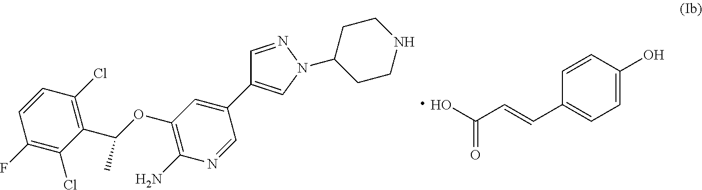 [SMILES: C[C@@H](Oc1cc(-c2cnn(C3CCNCC3)c2)cnc1N)c1c(Cl)ccc(F)c1Cl ; O=C(O)/C=C/c1ccc(O)cc1]

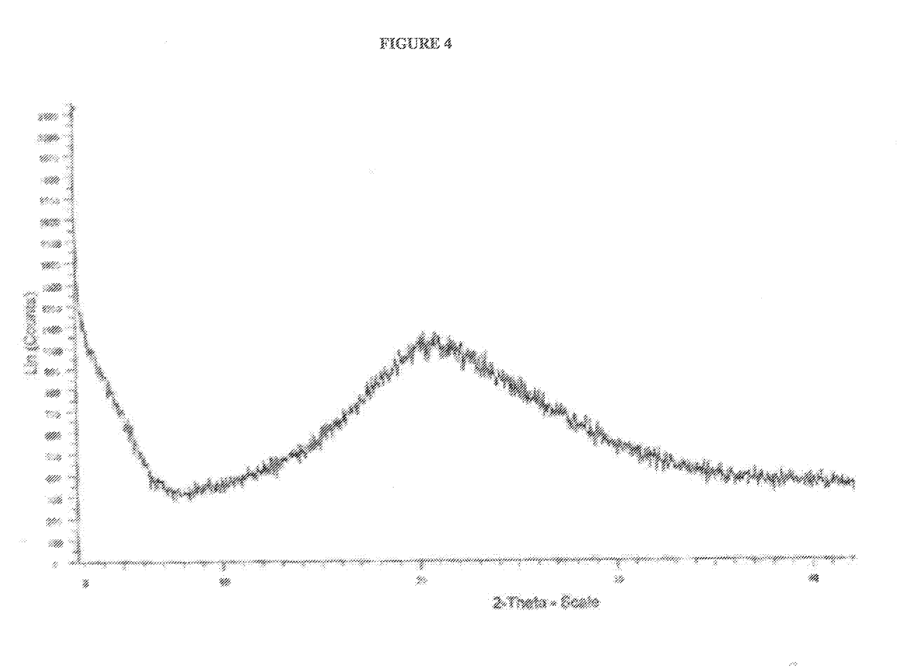

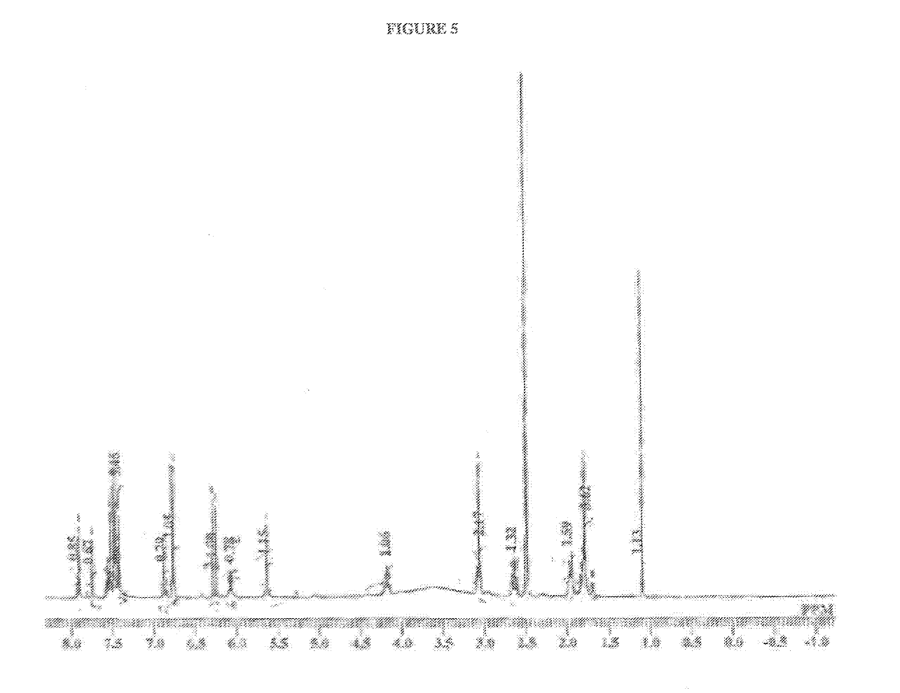

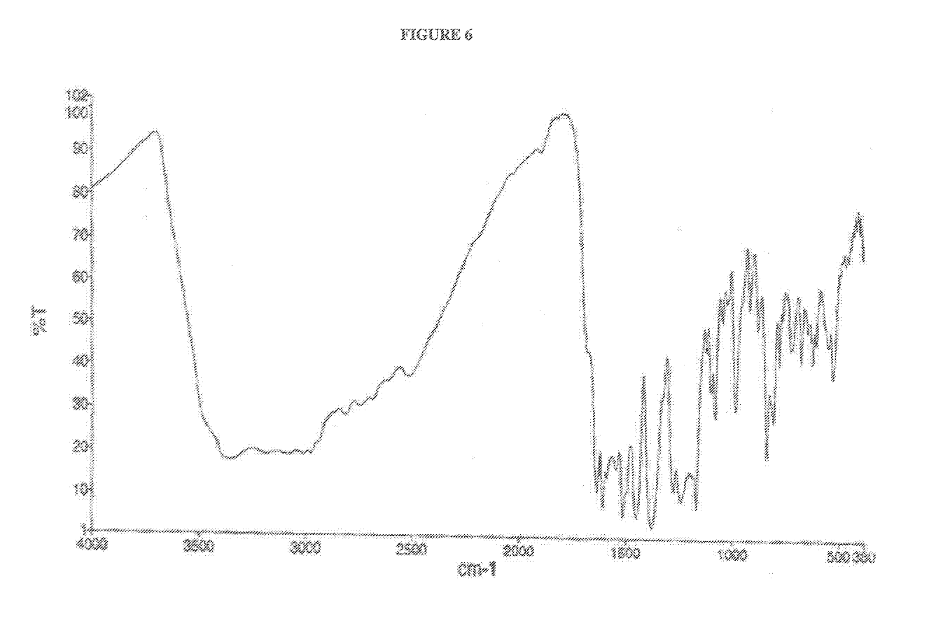

In a further embodiment of the present invention stable pharmaceutically acceptable substituted aryl acrylic acid addition salt of Crizotinib represented by Formula (Ib) i.e. Crizotinib Coumarate is characterized by:
a) X-ray powder diffraction pattern substantially according to FIG. 4;
b) ¹H NMR spectrum substantially according to FIG. 5;
c) IR absorption peaks, at approximately 3356 cm⁻¹, 1274 cm⁻¹, 1587 cm⁻¹, 1634 cm⁻¹, 1117 cm⁻¹ and 776 cm⁻¹ or IR spectral pattern substantially according to FIG. 6.

The new salt forms of Crizotinib as described by the present application have been found to be quite stable and easy to handle and store for longer time without any measurable change in their morphology and physicochemical characteristics, while retaining their characteristics within the defined limits. This offers advantages for large scale manufacturing in terms of handling, storage, shelf life and favorable impurity profile.

In another embodiment of the present application, it provides a process for preparing stable pharmaceutically acceptable substituted aryl acrylic acid addition salt of Crizotinib (I),

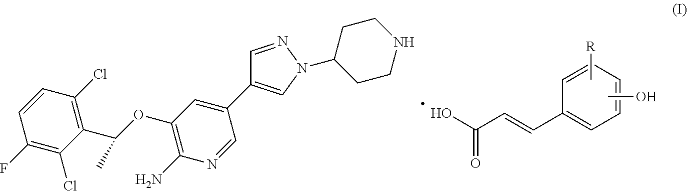

comprising the steps of:
a) providing a solution of Crizotinib base in an organic solvent at temperature of 20-30° C.;
b) adding substituted aryl acrylic acid of Formula (A) to the reaction mixture;

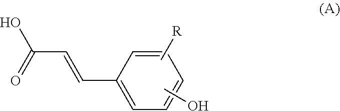

wherein, R is selected from —H, —OH, or —O—C₁₋₃alkyl;
c) extracting the solvent from reaction mixture;
d) treating the product obtained from step c) with another organic solvent;
e) isolating the stable pure crystalline pharmaceutically acceptable substituted aryl acrylic acid addition salt of Crizotinib (I).

The individual steps of the process according to the present invention for preparing stable pharmaceutically acceptable substituted aryl acrylic acid addition salt of Crizotinib (I) are detailed separately herein below.

Step a) comprises providing a solution of Crizotinib base in an organic solvent at temperature of 20-30° C.;

Crizotinib base from any source is added to an organic solvent which is taken 10-25 times v (in mL)/w (in g) w.r.t. weight of Crizotinib base. The reaction mass is stirred for 5 to 30 mins at temperature ranging between 20-30° C. depending upon the dissolution of Crizotinib base.

In an embodiment organic solvent used in this step may be chosen from C₁-C₄ alcohol for e.g. methanol, ethanol, n-propanol, iso-propanol, n-butanol, sec-butanol or tert-butanol; C₂-C₆ ether solvent for e.g. diethyl ether, diisopropyl ether, 1,4-dioxane, methyl tert-butyl ether or tetrahydrofuran; C₃-C₈ ketonic solvent for e.g. acetone, acetophenone, methyl isobutyl ketone or methyl isopropyl ketone; and, C₂-C₆ ester solvent for e.g. ethyl acetate, propyl acetate, isopropyl acetate or methyl acetate.

In a preferred embodiment the organic solvent used in this step is selected from C₁-C₄ alcohol for e.g. methanol, ethanol, n-propanol, iso-propanol, n-butanol, sec-butanol or tert-butanol.

Step b) comprises addition of substituted aryl acrylic acid of Formula (A) to the reaction mixture;

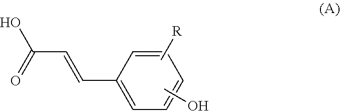

wherein, R is selected from —H, —OH, or —O—C₁₋₃ alkyl;

To the solution obtained in step a), substituted aryl acrylic acid of Formula (A) is added in the mole ratio of 0.5 to 2.0 w.r.t. to the amount of Crizotinib base. The reaction is carried out by stirring for 20-60 mins at a temperature ranging between 20-30° C., depending upon the progress of the reaction aimed at obtaining a clear reaction mixture. If required heating of reaction mixture may also be performed.

In a preferred embodiment of the present invention, substituted aryl acrylic acid of Formula (A) used in the present reaction is selected from Ferulic acid, o-Coumaric acid, m-Coumaric acid, p-Coumaric acid, Caffeic acid and the like.

Step c) comprises extracting the solvent from reaction mixture;

Solvent is extracted from the reaction mixture obtained from step b) to obtain a residue/reaction mass. Extraction of solvent from the reaction mixture can be performed by any conventional method known to the person having skill in the art. For e.g. in one of the specific embodiment of this invention, extraction of the solvent in this step is performed by distillation under reduced pressure conditions.

Extraction of solvent from the reaction mixture may be performed at a raised temperature of above 30° C. or at ambient room temperature. Extent of extraction of solvent from the reaction mixture may vary from complete extraction to partial solvent extraction. For e.g. in one of the specific embodiment of this application solvent ethanol was partially distilled out from the reaction mixture by use of vacuum, to get a reaction mixture with volume about one-third w.r.t the initial volume of the reaction mixture.

Step d) comprises treating the product obtained from step c) with another organic solvent;

The residue or reaction mass obtained from step c) is treated with another organic solvent. Organic solvent used in this step is selected from C₁-C₄ alcohol, C₂-C₆ ether solvent, C₃-C₈ ketonic solvent or C₂-C₆ ester solvent, wherein the organic solvent used in this step and the organic solvent used in step a) are not the same.

In an embodiment of the present invention, C₁-C₄ alcohol may be selected from methanol, ethanol, n-propanol, iso-propanol, n-butanol, sec-butanol or tert-butanol; C₂-C₆ ether solvent may be selected from diethyl ether, diisopropyl ether, 1,4-dioxane, methyl tert-butyl ether or tetrahydrofuran; C₃-C₈ ketonic solvent may be selected from acetone, acetophenone, methyl isobutyl ketone or methyl isopropyl ketone; and, C₂-C₆ ester solvent may be selected from ethyl acetate, propyl acetate, isopropyl acetate or methyl acetate.

In a preferred embodiment the organic solvent used in this step is selected from C₂-C₆ ether solvent for e.g. diethyl ether, diisopropyl ether, 1,4-dioxane, methyl tert-butyl ether or tetrahydrofuran.

Excess amount of organic solvent may be used in this reaction step, amount of organic solvent used ranges from 35-70 times v (in mL)/w (in g) w.r.t. weight of Crizotinib base initially taken. Organic solvent may be used in a single lot or in 2-5 batches. The reaction mass obtained in this step may optionally be subjected to heating to a temperature above 40° C. or cooling to a temperature below 10° C. The reaction mass may be subjected to stirring for time duration varying from 1 hr to 24 hrs.

In one of the embodiment of this invention, wherein the reaction mass obtained in this step is heated to a temperature above 40° C., after suitable duration of stirring, the reaction mass may be then cooled to a temperature below 30° C. In certain cases these steps of addition of organic solvent, heating reaction mass to a temperature above 40° C. and then cooling to a temperature below 30° C. may be repeated with the intermittently isolated product so as to achieve compliance to required final product characteristics and the stipulated impurity profile compliance.

Step e) comprises isolating the stable pure crystalline pharmaceutically acceptable substituted aryl acrylic acid addition salt of Crizotinib (I).

On completion of the reaction in step d), the reaction mass is filtered and washed again with the suitable organic solvent, which may be the same solvent that is used in step d). The solid material obtained is then dried at a temperature ranging between 45-60° C. thus providing the crystalline substituted aryl acrylic acid addition salt of Crizotinib (I) as end product. Drying may be performed under reduced pressure conditions for e.g. by use of vacuum.

Process of isolating substituted aryl acrylic acid addition salt of Crizotinib (I) comprise processes but not limited to conventional processes including scrapping, if required filtering from slurry and optionally further drying, which may be carried out at room temperature for the suitable durations.

The Crizotinib salt obtained may have Crizotinib base and substituted aryl acrylic acid in the ratio of 0.5 to 2.0 or any other ratio depending upon the amount of acid used in reaction of step b) and the actual reaction conditions utilized by the person skilled in the art. Substituted aryl acrylic acid addition salt of Crizotinib of this invention, may exist as a hydrated, solvated or a non-solvated form.

In one of the preferred embodiments of the present invention substituted aryl acrylic acid addition salt of Crizotinib, for e.g. Crizotinib Ferulate (la) and Crizotinib Coumarate (Ib) obtained as per the process of the present invention are found devoid of any crystal lattice and are adequately stable to handle and store for longer time without any significant or measurable change in their morphology and physicochemical characteristics. Substituted aryl acrylic acid addition salt of Crizotinib obtained according to the process of the present invention results in the final API purity by HPLC of more than 99% w/w.

The substituted aryl acrylic acid addition salt of Crizotinib (I) described herein may be characterized and analyzed by X-ray powder diffraction pattern (XRPD) on a Bruker AXS D8 Advance Diffractometer using X-ray source—Cu Kα radiation using the wavelength 1.5418 Å and lynx Eye detector. IR study was performed on Perkin Elmer Spectrum ES Version 10.03.03 instrument. Illustrative examples of analytical data for the Crizotinib Ferulate salt (Ia) and Crizotinib Coumarate salt (Ib) obtained in the Examples are set forth in the FIGS. 1-6.

In a further embodiment according to the specification, the invention also relates to a composition containing substituted aryl acrylic acid addition salt of Crizotinib (I) or a hydrate or solvate thereof. Substituted aryl acrylic acid addition salt of Crizotinib (I) is preferably selected from Crizotinib Ferulate or Crizotinib Coumarate.

The substituted aryl acrylic acid addition salt of Crizotinib (I) or a hydrate or solvate thereof obtained by the process of the present application may be formulated as solid compositions for oral administration in the form of capsules, tablets, pills, powders or granules. In these compositions, the active product is mixed with one or more pharmaceutically acceptable excipients. The drug substance can be formulated as liquid compositions for oral administration including solutions, suspensions, syrups, elixirs and emulsions, containing solvents or vehicles such as water, sorbitol, glycerin, propylene glycol or liquid paraffin.

The compositions for parenteral administration can be suspensions, emulsions or aqueous or non-aqueous sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g. using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable excipients used in the compositions comprising substituted aryl acrylic acid addition salt of Crizotinib (I) or a hydrate or solvate thereof, of the present application include, but are but not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, pre-gelatinized starch and the like; disintegrants such as starch, sodium starch glycolate, pregelatinized starch, Croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, waxes and the like. Other pharmaceutically acceptable excipients that are of use include but not limited to film formers, plasticizers, colorants, flavoring agents, sweeteners, viscosity enhancers, preservatives, antioxidants and the like.

Pharmaceutically acceptable excipients used in the compositions of substituted aryl acrylic acid addition salt of Crizotinib (I) or a hydrate or solvate thereof of the present application may also comprise to include the pharmaceutically acceptable carrier used for the preparation of solid dispersion, wherever utilized in the desired dosage form preparation.

Certain specific aspects and embodiments of the present application will be explained in more detail with reference to the following examples, which are provided by way of illustration only and should not be construed as limiting the scope of the invention in any manner.

### EXAMPLES

### Example-01

### Process for Preparation of Crizotinib Ferulate (Ia)

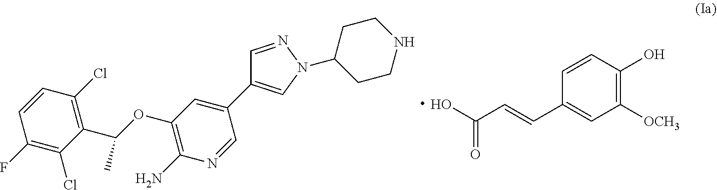 [SMILES: C[C@@H](Oc1cc(-c2cnn(C3CCNCC3)c2)cnc1N)c1c(Cl)ccc(F)c1Cl ; COc1cc(/C=C/C(=O)O)ccc1O]

7.5 mL ethanol was charged into a 100 ml 3-neck round bottomed flask at ˜25° C. 0.5 g Crizotinib base was added to it and the reaction mass was stirred for 15 mins, to get a clear solution. To this clear solution 0.23 g Ferulic acid was added and the reaction mixture was stirred for 30 mins. Then ethanol was completely distilled out from the reaction mixture by use of vacuum to get a residue.

To the residue obtained above, 2.0 mL methyl tert-butyl ether (MTBE) was added which was later distilled out under vacuum at temperature of ˜35° C. This step was repeated again with 2.0 mL MTBE and a semi-solid material was obtained. Then 12.5 mL MTBE was added to this semi-solid material and the reaction mass was heated to 55° C., this temperature being maintained for 1 hr. Then the reaction mass was cooled to 25° C. wherein stirring was maintained for 30 mins.

The product obtained was filtered, washed with with 2.0 mL MTBE and suck dried to obtain a wet solid. This wet solid was added to a 100 ml 3-necked round bottomed flask having 12.5 mL MTBE. The reaction mass was then heated to 55° C. and maintained for ˜1 hr. This was followed by cooling of the reaction mass to 25° C. wherein stirring was maintained for 30 mins. The product was filtered from the reaction mass and washed with 2.0 mL MTBE. The obtained solid material was then suck dried, unloaded and further dried under vacuum at 55° C. for 16 h to obtain 0.63 g of Crizotinib Ferulate (Ia) having X-ray powder diffraction pattern according to FIG. 1, IR spectral pattern according to FIG. 3 and melting range of 90.2-123.1° C.

Yield: 88.11%

¹H NMR (400 MHz, DMSO) δ 7.91-7.98 (d, 1H), 7.75 (d, 1H), 7.52-7.59 (m, 1H), 7.42-7.46 (m, 11H), 7.40-7.41 (d, 1H), 7.24 (d, 1H), 7.03-7.06 (dd, 1H), 6.87-6.88 (dd, 1H), 6.75-6.78 (d, 1H), 6.32-6.36 (d, 1H), 6.06-6.12 (q, 1H), 5.63 (s, 2H), 4.14-4.2 (m, 1H), 3.79 (s, 3H), 3.05-3.08 (m, 2H), 2.6-2.66 (m, 2H), 1.83-1.97 (m, 2H), 1.79-1.80 (d, 3H), 1.78-1.81 (m, 2H)

### Example-02

### Process for Preparation of Crizotinib Ferulate

7.5 mL Ethanol was charged into a 100 ml 3-neck round bottomed flask at ˜30° C. 0.5 g Crizotinib base was added to it and the reaction mass was stirred for 10 mins, to get a clear solution. To this clear solution 0.237 g ferulic acid was added and the reaction mixture was further stirred for 30 mins. Then ethanol was partially distilled out from the reaction mixture by use of vacuum, to get a reaction mixture with volume about one-third w.r.t the initial volume of the reaction mixture.

Then 20.5 mL MTBE was added to the reaction mass, followed by its cooling to ˜0° C. along with continuous stirring. The temperature of ˜0° C. and stirring were maintained for 24 hrs. Then at same temperature the product obtained was filtered and washed with with 4.0 mL MTBE to obtain a semi-solid material. The semi-solid material was then suck dried, unloaded and further dried under vacuum at 50° C. for 16 h to obtain 0.50 g of Crizotinib Ferulate (Ia) having X-ray powder diffraction pattern similar to FIG. 1, ¹H NMR similar to FIG. 2, IR spectral pattern similar to FIG. 3 and melting range of 88.2-125.7° C.

Yield: 70.42%

### Example-03

### Process for Preparation of Crizotinib Coumarate (Ib)

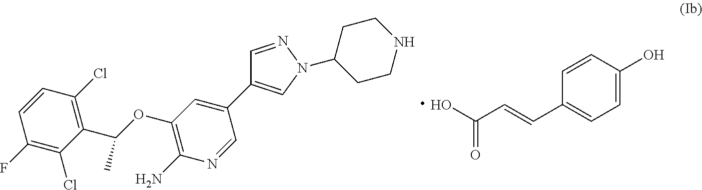 [SMILES: C[C@@H](Oc1cc(-c2cnn(C3CCNCC3)c2)cnc1N)c1c(Cl)ccc(F)c1Cl ; O=C(O)/C=C/c1ccc(O)cc1]

7.5 mL Ethanol was charged into a 100 ml 3-neck round bottomed flask at ˜30° C. 0.5 g Crizotinib base was added to it and the reaction mass was stirred for 10 mins, to get a clear solution. To this clear solution 0.2 g Coumaric acid was added and the reaction mixture was stirred for ˜30 mins. Then ethanol was distilled out under vacuum from the reaction mixture by to get a residue.

To the residue obtained above, 2.0 mL methyl tert-butyl ether (MTBE) was added which was later distilled out under vacuum. This step was repeated again with 2.0 mL MTBE and a semi-solid material was obtained. Then 12.5 mL MTBE was added to this semi-solid material and the reaction mass was heated to 55° C., this temperature being maintained for 1 hr. The reaction mass turned into a suspension which was then cooled to 25° C. wherein stirring was maintained for 30 mins.

The product obtained was filtered, washed with with 2.0 mL MTBE and suck dried to obtain a wet solid. This wet solid was added to a 100 ml 3-necked round bottomed flask having 12.5 mL MTBE. The reaction mass was then heated to 50° C. and maintained for ˜1 hr. This was followed by cooling of the reaction mass to 30° C. wherein stirring was maintained for 30 mins. The product was filtered from the reaction mass and washed with 2.0 mL MTBE. The obtained solid material was then suck dried, unloaded and further dried under vacuum at 50° C. for 16 h to obtain 0.59 g of Crizotinib Coumarate (Ib) having X-ray powder diffraction pattern according to FIG. 4, IR spectral pattern according to FIG. 5 and melting range of 90.5-125.3° C.

Yield: 86.76%

¹H NMR (400 MHz, DMSO) δ 7.91-7.92 (d, 1H), 7.75 (d, 1H), 7.52-7.54 (m, 1H), 7.50-7.52 (d, 1H), 7.487-7.50 (m, 1H), 7.45-7.46 (d, 1H), 7.408-7.43 (m, 1H), 6.87-6.88 (dd, 1H), 6.75-6.78 (d, 1H), 6.24-6.28 (d, 1H), 6.04-6.09 (q, 1H), 5.63 (s, 2H), 4.14-4.2 (m, 1H), 3.05-3.08 (m, 2H), 2.6-2.66 (m, 2H), 1.83-1.97 (m, 2H), 1.78-1.79 (d, 3H), 1.68-1.81 (m, 2H)

### Example-04

### Process for Preparation of Crizotinib Coumarate

2.0 g of Boc-Crizotinib was charged in to 20.0 mL DCM at ˜30° C., the reaction mass was stirred for 5-10 mins and then cooled to 10-15° C. to obtain clear solution. 14.0 mL IPA.HCl (15-20%) was added drop wise over the period of 20-30 min at 10-15° C. and the reaction mass was stirred 4 hrs at 25-30° C. After completion of reaction distilled out DCM and IPA completely to get thick mass. To this 30.0 mL of Water was added and stirred for 10 min to get clear solution. 20.0 mL of DCM was charged to the aqueous solution and stirred for 15-20 min. Separate the two layer and discarded DCM layer, repeat this process by adding 20.0 mL of DCM. The aqueous layer obtained was cooled to 10-15° C. and adjust the pH of the reaction mass between 11-13 by using 40% aqueous NaOH solution. To the reaction mass 30.0 mL of DCM was added to extract the product into DCM and stirred for 15-20 min. Separate the two layers again to the aqueous layer, 20.0 mL of DCM was added stirred for 15-20 min. Separate the two layers, combine two organic layers and washed with 10.0 mL of water. The obtained organic layer and dried over sodium sulphate then distilled out DCM to get crude Crizotinib base. Add 14.0 mL of fresh DCM and stirred for 5-10 min, reaction mass was cooled to 10-15° C. To this clear solution 0.50 g of p-Coumaric acid was added at 10-15° C. and stirred for 5-6 hrs at 10-15° C. 28.0 mL of MTBE was added to the reaction mass at 10-15° C. over the period of 10-15 min. and stirring were maintained for 1 h. Then at the same temperature the product was filtered and washed with 4.0 mL of MTBE. The obtained solid material was suck dried, unloaded and further dried under vacuum at 50° C. for 16 h to obtain 1.6 g of Crizotinib Coumarate (Ib).

Yield: 71.7%

HPLC: 99.60%.

### Example-05

### Process for Preparation of Crizotinib Ferulate Salt

2.0 g of Boc-Crizotinib was added in to a round bottomed flask containing 20.0 mL DCM at ˜30° C., the reaction mass was stirred for 5-10 mins and was cooled to 10-15° C. to obtain a clear solution. 14.0 mL IPA.HCl (15-20%) was added drop wise over the period of 20-30 min at 10-15° C. and the reaction mass was stirred 4 hrs at 25-30° C. After completion of reaction distilled out DCM and IPA completely to get thick mass. To this 30.0 mL of Water was added and stirred for 10 min to get clear solution. 20.0 mL of DCM was charged to the aqueous solution and stirred for 15-20 min. Separate two layer and discarded DCM layer, again 20.0 mL of DCM was charged to the aqueous layer and stirred for 15-20 min. Separate two layers and discarded DCM layer. The aqueous layer obtained was cooled to 10-15° C. and pH of the reaction mass was adjusted in between 11-13 using 40% aqueous NaOH. To the reaction mass 30.0 mL of DCM was added to extract the product into DCM and stirred for 15-20 min. Separate the two layers again taken aqueous layer, 20.0 mL of DCM was added stirred for 15-20 min. Separate the two layers and combine two organic layers and washed with 10.0 mL of water. The organic layer was dried over sodium sulphate then distilled out DCM to get crude Crizotinib base. Add 14.0 mL of fresh DCM and stirred for 5-10 min, reaction mass was cooled to 10-15° C. To this clear solution 0.60 g of ferulic acid was added at 10-15° C. and stirred for 5-6 hrs at 10-15° C. 28.0 mL of MTBE was added to the reaction mass at 10-15° C. over the period of 10-15 min. and stirring were maintained for 1 h. Then at the same temperature the product was filtered and washed with 4.0 mL of MTBE. The obtained solid material was suck dried, unloaded and further dried under vacuum at 50° C. for 16 h to obtain 1.7 g of Crizotinib Ferulate (Ia).

Yield: 72.6%

While the foregoing provides a detailed description of the preferred embodiments of the invention, it is to be understood that the descriptions are illustrative only of the principles of the invention and not limiting. Furthermore, as many changes can be made to the invention without departing from the scope of the invention, it is intended that all material contained herein be interpreted as illustrative of the invention and not in a limiting sense.

We claim:

1. Stable pharmaceutically acceptable substituted aryl acrylic acid addition salt of Crizotinib represented by Formula (I) 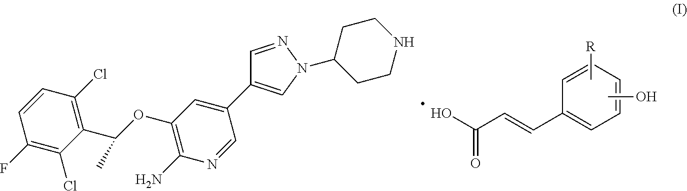
wherein, R is selected from —H, —OH, or —O—C₁₋₃alkyl; or a hydrate or solvate of the said salt.

2. Stable pharmaceutically acceptable substituted aryl acrylic acid addition salt of Crizotinib (I) or a hydrate or solvate thereof, according to claim 1 wherein, R is selected from —H, —OH, or —OCH₃.

3. Stable pharmaceutically acceptable substituted aryl acrylic acid addition salt of Crizotinib (I) according to claim 1 wherein the said salt is represented by Formula (Ia), 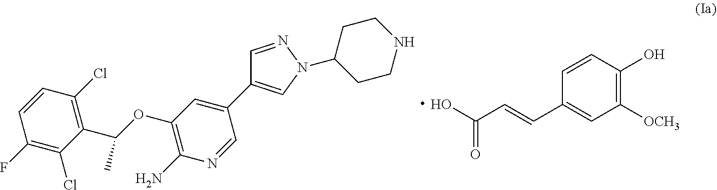 [SMILES: C[C@@H](Oc1cc(-c2cnn(C3CCNCC3)c2)cnc1N)c1c(Cl)ccc(F)c1Cl ; COc1cc(/C=C/C(=O)O)ccc1O]
and is characterized by X-ray powder diffraction pattern as per FIG. 1 and IR absorption peaks, at approximately 3377 cm⁻¹, 1321 cm⁻¹, 1593 cm⁻¹, 1634 cm⁻¹, 1124 cm⁻¹ and 776 cm⁻¹.

4. Stable pharmaceutically acceptable substituted aryl acrylic acid addition salt of Crizotinib (I) according to claim 1 wherein the said salt is represented by Formula (Ib), 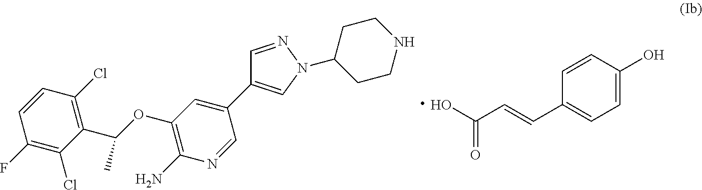 [SMILES: C[C@@H](Oc1cc(-c2cnn(C3CCNCC3)c2)cnc1N)c1c(Cl)ccc(F)c1Cl ; O=C(O)/C=C/c1ccc(O)cc1]
and is characterized by X-ray powder diffraction pattern as per FIG. 4 and IR absorption peaks, at approximately 3356 cm⁻¹, 1274 cm⁻¹, 1587 cm⁻¹, 1634 cm⁻¹, 1117 cm⁻¹ and 776 cm⁻¹.

5. Stable pharmaceutically acceptable substituted aryl acrylic acid addition salt of Crizotinib (I) according to claim 1, wherein the said salt form or a hydrate or solvate thereof is optionally in a crystalline form or in amorphous form.

6. A process for preparing stable pharmaceutically acceptable substituted aryl acrylic acid addition salt of Crizotinib (I), 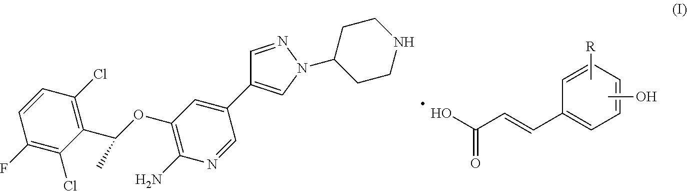
comprising the steps of:
a) providing a solution of Crizotinib base in an organic solvent at temperature of 20-30° C.;
b) adding substituted aryl acrylic acid of Formula (A) to the reaction mixture; 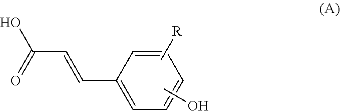
wherein, R is selected from —H, —OH, or —O—C₁₋₃alkyl;
c) extracting the solvent from reaction mixture;
d) treating the product obtained from step c) with another organic solvent; and
e) isolating the stable pure crystalline pharmaceutically acceptable substituted aryl acrylic acid addition salt of Crizotinib (I).

7. A process for preparing stable pharmaceutically acceptable substituted aryl acrylic acid addition salt of Crizotinib (I) according to claim 6, wherein organic solvent is selected from C₁-C₄ alcohol, C₂-C₆ ether solvent, C₃-C₈ ketonic solvent or C₂-C₆ ester solvent, provided that the organic solvent used in step a) and step d) are not the same.

8. A process for preparing stable pharmaceutically acceptable substituted aryl acrylic acid addition salt of Crizotinib (I) according to claim 7, wherein C₁-C₄ alcohol is selected from methanol, ethanol, n-propanol, iso-propanol, n-butanol, sec-butanol or tert-butanol; C₂-C₆ ether solvent is selected from diethyl ether, diisopropyl ether, 1,4-dioxane, methyl tert-butyl ether or tetrahydrofuran; C₃-C₈ ketonic solvent is selected from acetone, acetophenone, methyl isobutyl ketone or methyl isopropyl ketone; and, C₂-C₆ ester solvent is selected from ethyl acetate, propyl acetate, isopropyl acetate or methyl acetate.

9. A process for preparing stable pharmaceutically acceptable substituted aryl acrylic acid addition salt of Crizotinib (I) according to claim 6, wherein step d) optionally involves heating of the reaction mass to a temperature above 40° C. or cooling to a temperature below 10° C.

10. A pharmaceutical composition comprising stable pharmaceutically acceptable substituted aryl acrylic acid addition salt of Crizotinib (I) or a hydrate or solvate thereof, together with one or more pharmaceutically acceptable excipients.
